(19) United States
(12) Patent Application Publication
(10) Pub. No.: US 2012/0251467 A1
(43) Pub. Date: Oct. 4, 2012

(54) FULVIC ACID COMPOSITIONS AND THEIR USE

(76) Inventor: Stephen William Leivers, Wymondham (GB)

(21) Appl. No.: 13/392,620

(22) PCT Filed: Aug. 27, 2010
(86) PCT No.: PCT/GB2010/001641

§ 371 Date: Jun. 8, 2012

(30) Foreign Application Priority Data
Aug. 27, 2009 (GB) 0914966.7

(51) Int. Cl. A61K 8/49; A61Q 11/00; A61P 31/00

(52) U.S. Cl. 424/54

(57) ABSTRACT

Carbohydrate-derived fulvic acid (CHD-FA), a salt, ester or derivative thereof for use in the prevention, treatment or inhibition of infection by oral pathogens in a mammal, compositions, particularly compositions suitable for oral administration, comprising the same.

### FIELD OF INVENTION

This invention relates to a use of fulvic acid in the prevention and treatment of infection by oral pathogens.

### BACKGROUND OF THE INVENTION

Fulvic acid (FA) is a humic substance along with humic acid and humin that is formed during the decay of organic matter (MacCarthy et al., 1985). These substances are characterised on the basis of their solubility in water as a function of pH and FA is the fraction that is soluble in water under all pH conditions. In general, FA is also lower in molecular size and weight and lower in colour intensity than the humic acids.

Although soil and water naturally contain low levels of FA, this is difficult to isolate. Wet oxidation of bituminous coal, as described in U.S. Pat. No. 4,912,256 yields oxifulvic acids. Use of these oxifulvic acids for treatment of inflammation, acne, eczema, bacterial, fungal and viral infections has been described in U.S. Pat. Nos. 4,999,202 and 5,204,368 and International Patent Publication No. WO00/19999. However, oxifulvic acids contain high concentrations of heavy metals, including mercury, aluminium, chromium, lead and cadmium and are therefore not appropriate for use in medical, pharmaceutical and cosmetic preparations.

Carbohydrate sources such as saccharides including glucose, sucrose and fructose, starches and cellulose can also be treated by wet oxidation and produce a carbohydrate derived fulvic acid composition. This carbohydrate derived fulvic acid is suitable for use in medical, pharmaceutical and cosmetic preparations as it contains only a low content of the harmful elements. International Patent Publication No. WO2007/125492 describes the carbohydrate derived fulvic acid composition and a method for producing the composition (hereinafter referred to as CHD-FA).

### DISCLOSURE OF THE INVENTION

It has been found that fulvic acid as described and claimed in International Patent Publication No. WO 2007/125492 (CHD-FA), a salt, ester or derivative thereof is effective in preventing, treating or inhibiting infection by oral pathogens in mammals.

Thus, the invention provides, according to one aspect, CHD-FA, a salt, ester, or derivative thereof for use in the prevention, treatment or inhibition of infection by oral pathogens in a mammal, which may be human or animal.

The CHD-FA, salt, ester or derivative thereof may be administered in an amount from about 8% to 60% v/v of CHD-FA in an oral formulation. Preferably, the amount is about 35% v/v of CHD-FA in an oral formulation.

The CHD-FA, salt, ester or derivative thereof can have any pH, from acid to basic. For example, the pH of the CHD-FA can be raised by converting the acid into a salt, such as the sodium or potassium salt. This may be achieved by adding a suitable hydroxide to the CHD-FA.

According to a further embodiment of the invention, there is provided a composition comprising CHD-FA, salt, ester or derivative thereof and a suitable carrier for oral administration.

The composition may be in the form of a mouthwash, toothpaste, denture cleanser, impregnated dental floss or dental tape, endodontic irrigant or the like. The CHD-FA, salt, ester or derivative thereof may be an active ingredient or an adjuvant. The composition may further comprise flavourants and the like.

According to a further aspect of the invention, there is provided a method of preventing, treating or inhibiting infection by oral pathogens in a mammal including the step of administering an effective amount of the CHD-FA, salt, ester or derivative thereof to the mammal.

The administration will be oral. For human administration, the CHD-FA, salt, ester or derivative thereof may be formulated into a form such as a mouthwash, toothpaste, denture cleanser, impregnated dental floss or dental tape, endodontic irrigant or the like. The CHD-FA, salt, ester or derivative thereof may be the active ingredient or an adjuvant. The formulated form may further comprise flavourants and the like.

### BRIEF DESCRIPTION OF THE DRAWINGS

FIG. 1 shows the effectiveness of CHD-FA in killing oral biofilms in comparison to other treatments i.e. Tea Tree Oil and the commercially available products, Oral B and Corsodyl.

FIG. 2 shows the relative lack of toxicity of CHD-FA when applied to the OKF6 cell line.

FIG. 3 shows the immuno-modulatory effect of CHD-FA on IL-6 and IL-8.

FIG. 4 shows the relative lack of toxicity of CHD-FA compared to Chlorhexadine, an ingredient commonly used to treat oral pathogens.

### DESCRIPTION OF PREFERRED EMBODIMENTS

CHD-FA, a salt, ester or derivative thereof for use in a method of preventing, treating or inhibiting infection by oral pathogens in mammals is described herein.

The optimal dose of the CHD-FA, a salt, ester or derivative thereof is an amount of about 35% of CHD-FA in an oral formulation maintained in the mouth for about 60 seconds. However a range of from about 8% to about 60% v/v CHD-FA in an oral formulation maintained in the mouth from about 30 seconds to about 60 seconds could also be used.

The CHD-FA, salt, ester or derivative thereof can have any pH, from acid to basic. For example, the pH of the CHD-FA can be raised by converting the acid into a salt, such as the sodium or potassium salt. This may be achieved by adding a suitable hydroxide to the CHD-FA.

The administration would generally be oral. Typically, the CHD-FA, salt, ester or derivative thereof would be formulated into a form such as a mouthwash, toothpaste, denture cleanser, impregnated dental floss or dental tape, or endodontic irrigant or the like, with the CHD-FA, salt, ester or derivative thereof as the active ingredient, or an adjuvant. Additional ingredients, including flavourants may also be included in the oral formulation.

An in vitro study was conducted to evaluate the antimicrobial activity of CDH-FA against a range of oral pathogens associated with caries, periodontal disease, endodontic infection and soft tissue infections. In addition, the toxicological properties of the compound were evaluated.

The following example is for the purpose of illustration only and is not to be construed as limiting on the invention in any way.

### EXAMPLES

### Methods

A panel of oral pathogens, including Gram-positive, Gram-negative and yeasts were grown planktonically and tested with CHD-FA using standardised CLSI methodology. In addition, biofilm populations were grown using a 96-well peg plate method, challenged with CHD-FA, and the sessile MIC's evaluated. Static and cidal activity was assessed. A TR146 oral epithelial cell line was used to assess the toxicity of CHD-FA using both metabolic (XTT) and LDH assays.

### Results

CHD-FA (0.5% active matter) was effective against the entire panel of planktonic Gram positive, Gram negative bacteria and yeasts, which included Streptococcus mutans, Porphymonas gingivalis and Candida albicans, and it was shown to exhibit cidal activity.

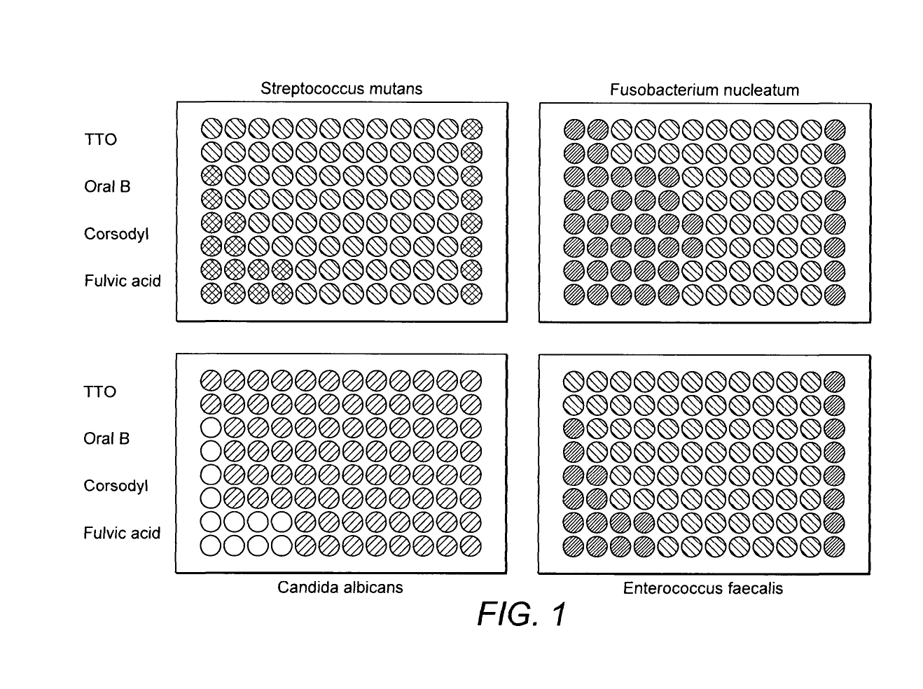

Against biofilms the CHD-FA was less effective, with a range of activity of 2-≧4% active matter. However, CHD-FA was shown to be more effective in killing oral biofilms in comparison to other treatments i.e. Tea Tree Oil and the commercially available products, Oral B and Corsodyl as illustrated in FIG. 1.

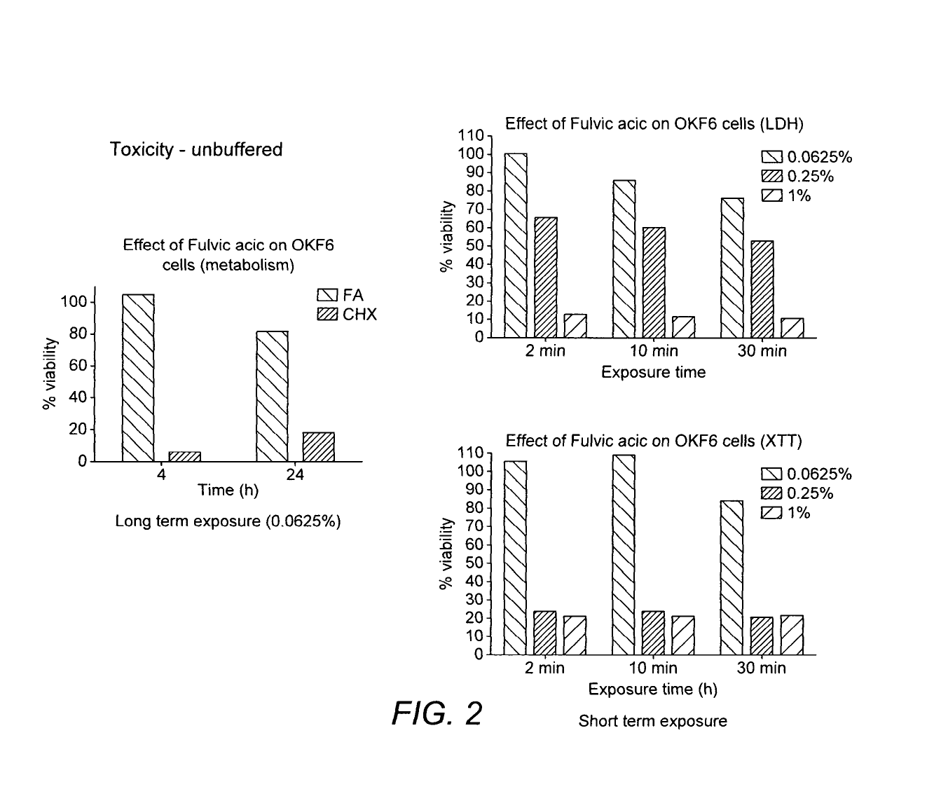

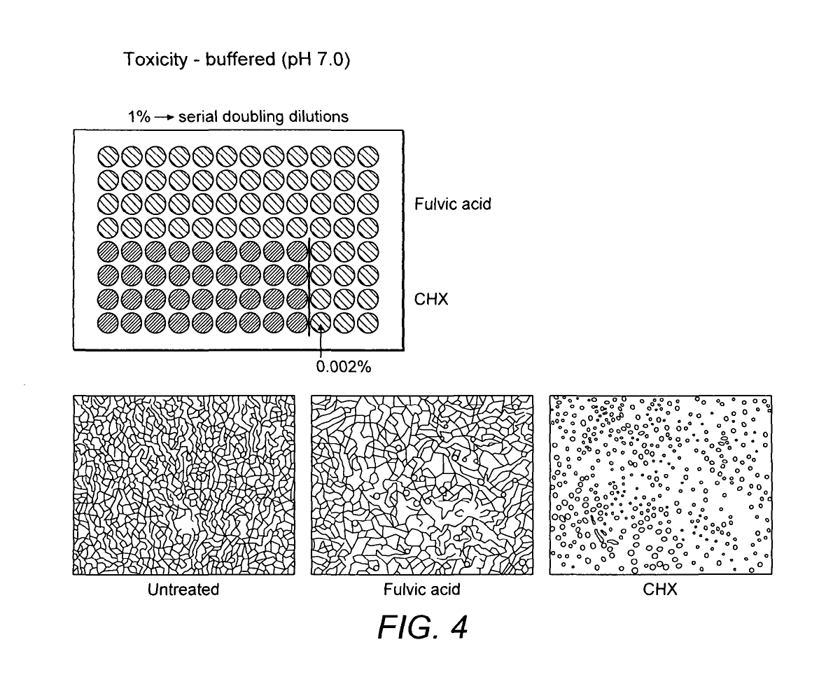

In addition, CHD-FA was shown to be relatively non-toxic when applied to the OKF6 cell line (see FIG. 2). When the cell line was exposed a concentration of 0.5% active matter CHD-FA, the planktonic MIC, it did not exhibit any cellular toxicity by either assay. Furthermore, CHD-FA is relatively non-toxic compared to Chlorhexadine, an ingredient commonly used to treat oral pathogens (see FIG. 4).

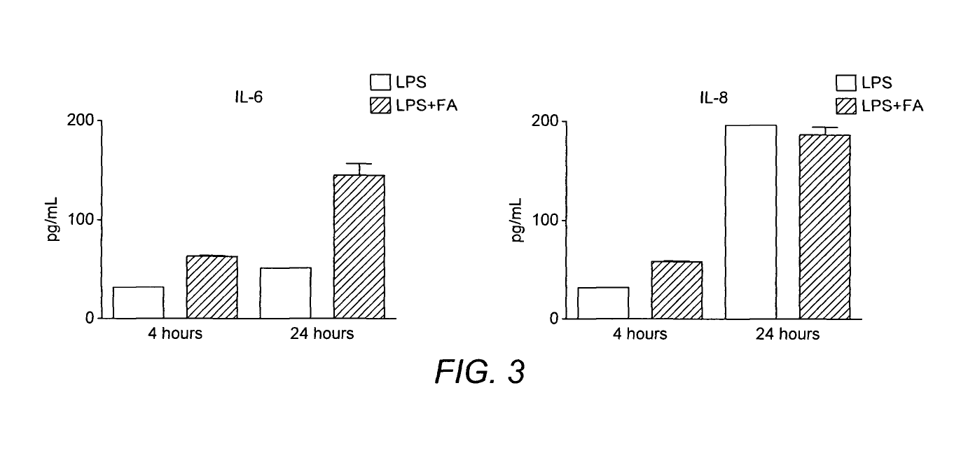

As indicated in FIG. 3, CHD-FA was shown to have an immune-modulatory effect on the cytokine IL-6 and the chemokine IL-8 which are often associated with inflammation.

As shown in Table 1, when the efficacy of CHD-FA is compared to the natural product Tea Tree Oil, or existing over-the counter products, Oral B and Corsodyl, we see that CHD-FA is superior to all the other treatments for a range of oral pathogens.

The values in percent in the Figures refers to % active matter.

TABLE 1
Efficacy of CHD-FA against a range of oral pathogens

| | NATURALS | | | | | | OTC's | | | | | |
|---|---|---|---|---|---|---|---|---|---|---|---|---|
| | DRUG | | | | | | | | | | | |
| | Fulvic Acid % | | | Tea Tree Oil % | | | Oral B % | | | Corsodyl ® % | | |
| Bacterium | PMIC | PMBC | SMIC | PMIC | PMBC | SMIC | PMIC | PMBC | SMIC | PMIC | PMBC | SMIC |
| S. sanguinis | 0.0625 | 0.0625 | 0.25 | >10 | >10 | 10 | 0.3125 | 5 | 10 | 0.3125 | 0.625 | 5 |
| S. salivarius | 0.0625 | 0.125 | 0.25 | >10 | >10 | >10 | >10 | >10 | 10 | 0.3125 | 0.3125 | 5 |
| S. mutans | 0.125 | 0.25 | 0.25 | >10 | >10 | >10 | 2.5 | >10 | 10 | 0.3125 | 0.625 | 5 |
| E. faecalis | 0.125 | 0.25 | 0.25 | >10 | >10 | >10 | 1.25 | 2.5 | 10 | 0.3125 | >10 | 5 |
| Aggregatibacter | 0.0625 | 0.125 | 0.25 | >10 | >10 | >10 | 0.625 | 5 | >10 | 0.625 | 0.625 | 10 |
| actinomycetemcomitans | | | | | | | | | | | | |
| F. nucleatum | 0.0625 | 0.0625 | 1.25 | >10 | >10 | 5 | 2.5 | 2.5 | 0.625 | 2.5 | 2.5 | 0.3125 |
| P. gingivalis | 0.03125 | 0.25 | 0.25 | >10 | >10 | 10 | 10 | >10 | 1.25 | 1.25 | 2.5 | 1.25 |
| C. albicans | 0.125 | 0.125 | 0.25 | 0.625 | 2.5 | >10 | 0.156 | 0.156 | 5 | 0.625 | 1.25 | 5 |
| C. glabrata | 0.125 | 0.125 | 0.25 | 0.625 | >10 | 0.25 | 0.156 | 0.3125 | 2.5 | 0.625 | 1.25 | 5 |
| C. tropicalia | 0.03125 | 0.125 | 0.25 | 0.3125 | >10 | 5 | 0.078 | 0.078 | 2.5 | 0.3125 | 0.3125 | 2.5 |

### Conclusions

This study has shown that the natural antimicrobial CHD-FA is non-toxic and has cidal activity against a range of microbes associated with a variety of oral diseases. Therefore, as a mouthwash, this product has potential as an adjunct to mechanical disruption to minimise the microbial burden in the oral cavity, subject to further studies.

1. An oral pharmaceutical composition for preventing, treating or inhibiting an infection by an oral pathogen, comprising:
(a) carbohydrate-derived fulvic acid (CHD-FA), a salt, ester or derivative thereof as active ingredient; and
(b) a pharmaceutically acceptable carrier for oral administration of the active ingredient.

2.-3. (canceled)

4. The pharmaceutical composition according to claim 3, in which the active ingredient is present in an amount from about 8% to 60% v/v of the composition.

5. The pharmaceutical composition according to claim 4, in which the active ingredient is present in an amount of about 35% v/v of the composition.

6. (canceled)

7. The pharmaceutical composition according to claim 1 that is formulated as a mouthwash, a toothpaste, an impregnated dental floss or dental tape, or an endodontic irrigant.

8.-12. (canceled)

13. The pharmaceutical composition according to claim 1, further comprising a flavorant.

14. A method of preventing, treating or inhibiting infection by an oral pathogen in a mammalian subject in need thereof, comprising administering to the subject an effective amount of a pharmaceutical composition that comprises:
(a) CHD-FA, or a salt, ester or derivative thereof; and
(b) a pharmaceutically acceptable carrier for oral administration.

15. The method according to claim 14, wherein the subject is a human.

16. The method according to claim 14, wherein the infection is in the form of an oral biofilm.

17. The method according to claim 14, wherein the CHD-FA, or the salt, ester or derivative thereof is present in an amount from about 8% to 60% v/v of the composition.

18. The method according to claim 14, wherein the CHD-FA, or the salt, ester or derivative thereof is present in an amount of about 35% v/v of the composition.

19. The method according to claim 14, wherein the pharmaceutical composition further comprises a flavorant.

20. The method according to claim 14, wherein the pharmaceutical composition is formulated as a mouthwash and is administered by rinsing the oral cavity with the mouthwash.

21. The method according to claim 14, wherein the pharmaceutical composition is formulated as a toothpaste and is administered by brushing the teeth with the toothpaste.

22. The method according to claim 14, wherein the pharmaceutical composition is formulated as impregnated dental floss or dental tape and is administered by flossing of teeth and gums.

23. The method according to claim 14, wherein the pharmaceutical composition is formulated as an endodontic irrigant administered as part of an endodontic procedure.
